SEQUENCE LISTING (13 sequences)

SEQ ID NO: 1 - DNA, 4372 bp, Homo sapiens
  CDS (357)..(1727)
aaacttggagagacgcaggacaggatcccggcggcagaaggacggagagaaaggggaccccgggacgggaaaggcgcagagcaggcgcgggcggcggcggcggcggggcagggcagggcgggcgtcccggcagagggcgcgcggtcgccctgtcgccctccgccccgccggggtcacagtgccccctccctcgcgccctagccgccctgccgggctatttttacgcgcggacaccggacaccggacaccgggctggggcggcggcggcggcggccgaggcggccgaggcggggccgcaccggggccgggcgtcggggccacacgtcggttcgcgggtcgccggggctgcgcgcgccatggagccgcggtgcccgccgccgtgcggctgctgcgagcggctggtgctcaacgtggccgggctgcgcttcgagacgcgggcgcgcacgctgggccgcttcccggacactctgctaggggacccagcgcgccgcggccgcttctacgacgacgcgcgccgcgagtatttcttcgaccggcaccggcccagcttcgacgccgtgctctactactaccagtccggtgggcggctgcggcggccggcgcacgtgccgctcgacgtcttcctggaagaggtggccttctacgggctgggcgcggcggccctggcacgcctgcgcgaggacgagggctgcccggtgccgcccgagcgccccctgccccgccgcgccttcgcccgccagctgtggctgcttttcgagtttcccgagagctctcaggccgcgcgcgtgctcgccgtagtctccgtgctggtcatcctcgtctccatcgtcgtcttctgcctcgagacgctgcctgacttccgcgacgaccgcgacggcacggggcttgctgctgcagccgcagccggcccgttccccgctccgctgaatggctccagccaaatgcctggaaatccaccccgcctgcccttcaatgacccgttcttcgtggtggagacgctgtgtatttgttggttctcctttgagctgctggtacgcctcctggtctgtccaagcaaggctatcttcttcaagaacgtgatgaacctcatcgattttgtggctatccttccctactttgtggcactgggcaccgagctggcccggcagcgaggggtgggccagcaggccatgtcactggccatcctgagagtcatccgattggtgcgtgtcttccgcatcttcaagctgtcccggcactcaaagggcctgcaaatcttgggccagacgcttcgggcctccatgcgtgagctgggcctcctcatctttttcctcttcatcggtgtggtcctcttttctagcgccgtctactttgccgaagttgaccgggtggactcccatttcactagcatccctgagtccttctggtgggcggtagtcaccatgactacagttggctatggagacatggcacccgtcactgtgggtggcaagatagtgggctctctgtgtgccattgcgggcgtgctgactatttccctgccagtgcccgtcattgtctccaatttcagctacttttatcaccgggagacagagggcgaagaggctgggatgttcagccatgtggacacgcagccttgtggcccactggagggcaaggccaatggggggctggtggacggggaggtacctgagctaccacctccactctgggcacccccagggaaacacctggtcaccgaagtgtgaggaacagttgaggtctgcaggacctcacacctccctagagggagggagggagggcagggtggagggcaaggctggggggaggggattgggtttaggaagagctaggttaagtcataacgagtggggaaacactgagtcttgttgggtcttgggttgtgtggtttggtagctcctgtgggtacctcctgaagcagcagcgaatggcaatgggttgtgttgtgttaatgaagactcaattggttcatattactctgagttgtgcaaagctcatggagccttttggggtagtgttgagataggtttggtcgtatcattttgtgagtttcctaggtcagtgttgggtttggttgggttgtgagtctgggatagtgtggtccagctgcattgtgtaggattctgtggtttggtgggtcccctagggccatgttgggtcaagttagatggtcccccatggcattgttgagatcgaatgtgtgtggtgttaagtttcgttgagacatggtggaaattgtgtagctctgtgattcttccaggggcatgttattttaggttctgtgaacttgcgagtcatgtagaaatgtgaagagtccagtggtagaatttgagctttctaggtcacattgggttaagtttgtatgaccaaatgaatcttgtagggttctgttgggcttaactgtgtagaggtgtgtggctggacatttttcgtggccacagcgagttgagttgtgttgaattgtacaaccatatgagccttgtaaggccagttcagttgggtcatgccactgtttgagtctcatagggccatgctgaattgagttccattgagttgtgtcactatgtgagtcctacaggaagttgggttgagttggactgtgcgaacgagttccatagggccacatcgggctgttttgcatttagtggtagcaccaggacccaaaggaaatagcagtggggaagcatcatgtatctgggagcatgcagtggcgagggctctgggaggtgtgccgagctggctccccagctcgctgtagggggcgggactggattctgtatccatgggattgggtgttcatccagaggcgactgggtaaattaggaagaggtggatgctcctcctgtttaccccacatccacttcattgtgctgttcactcccattctcccctacagttttatgctcagacatggaggtcagagccacaagggaaaggggagagggggagaaaactgtactctgtccagacatgatagagggacagagccaaaaggatagagaaagagacccagaaaaaggaagaggtggaaacccagagagacagagacccaaagggagagaaacagagactcagggagagggagacaatgacctggagggtggggtatggcagagacgcagaagagaggaacagaaatccagagtggggagacagagaccaagagcaggggatagaagccgggcgaagtggcccatgcctgtaatctcagcactctgggagaccgaggaagggggattgattgaggccaggagttcaagaccagcctgggcaacatggtgagaccccatctctacaaaaaatacaaaaattagctgagtgtggtggcacatgcctgtgatcccagctactcaggaagctgaggcagaaagatcccttgaccctgagaggtagaggctgcattgagccatgattgcaccactgcactccagcctgggcaacagagggagcccccgtctcaacaaacaaacaaaaagagccagtgggggagggagggacagagacccagagggcagcgtcagacacccagagttggagacagaacaacagagtctcagggaaagagaaccacaatagaaaaaggcagaaaaggccgggcgcggtggctcatgcctgtaaccccagcactttgggaggccgaggtgggcaaattacgaggtcaggagatccagaccatcctggctaacacggtgaaaccccgtctctactaaaaatacaaaaaaattagccgggtgcggtggcgggcacctgtagtcccagctactcgggaggctgaggcaggagaatggcatgaacctgggaggcggagcttgcagtgagccgagattgcgccactgcactccagcctgggcagcagagcaacactctgtctcaaaaaaaaaaagaaaagaaaagaaaaagccagaaaaagttggtgcccctgaacccaagagtgatgtacagtctattccatagaatcacagaacaatcctgaaccaggcctgtcacctaccctccctgcagctcaggaaggctgtcagacaggctgggggcctcactctgttttccaggggagaaacctgagtctcagagcaggggagtggcttcccaaggtctcacagcttgtccccaggggccaggcaggctgtctgtctgcttcacatgtccccatcagcctgctgggacacacgggtcctcctgagtcccgtagcctcatttcttacagacggggaaactgaggctcagagcagcaggtgttacccaaggtcacaaggccgaacattttcagaaatctttcagaactcaaagggcatttagaggaagaaggctgaaatcactaacacatatagggcttcctttggtatcaaagtactttacttgggttaatttatta

SEQ ID NO: 2 - protein, 456 aa, Homo sapiens [sequence omitted]

SEQ ID NO: 3 - DNA, 706 bp, Homo sapiens
  misc_feature: N=A,T,G or C
  ref <301>: AUTHORS: Zabarovsky et al.
  ref <302>: TITLE: NotI clones in the analysis of the human genome
  ref <303>: JOURNAL: Nucleic Acids Research
  ref <304>: VOLUME: 28
  ref <305>: ISSUE: 7
  ref <306>: PAGES: 1635- 1639
  ref <307>: DATE: 2000-__-__
  ref <308>: DATABASE ACCESSION NUMBER: GenBank/AQ939522
  ref <309>: DATABASE ENTRY DATE: 2000-04-03
gcggccgcgttgaacgagccaggcctggcttccaaggtcccggcacgcgagtgcaaggcaggatcctgggaggaagtctgggataaccaggtcacggagggccttggcgcagcggctactgaccaatgtgtaaagaggagctgggtatttaaatgatgattaaggctgtccccgtgtcctagccccagcctgaccctccctgaacactttcctccctgcagttccccgctcggctgaatggctccagccaaatgcctggaaatccaccccgcctgcccttcaatgacccgttcttcgtggtggagacgctgtgtatttgttggttctcctttgagctgctggtacgccttctggtctgtccaagcaaggctatcttcttcaagaacgtgatgaacctcatcgattttgtggctatccttccctactttgtggcactgggcacccgagctggcccggcagcgaggggtgggccancaggccatgtcacttggccattcttgagaagtatccgattggtgcgtgtnttccggaatcttcaagctgtccccgnactnaaaangggccttnnaattntttgggccaaanccnttngggcttcattgcgttgaagctggggcctccctaatcttttttcctctttcattcggtggtgggcctcttttttagccgccctctacttttgccgaagttgacccgggtgggacct

SEQ ID NO: 4 - DNA, 1599 bp, Mus musculus
  ref <301>: AUTHORS: Kalman et al.
  ref <302>: TITLE: Genomic Organization, Chromosomal Localization, Tissue Distribution, and Biophysical Characterization of a Novel Mammalian Shaker-related Voltage-gated Potassium Channel, Kv1.7
  ref <303>: JOURNAL: Journal of Biological Chemistry
  ref <304>: VOLUME: 273
  ref <305>: ISSUE: 10
  ref <306>: PAGES: 58515857
  ref <307>: DATE: 1998-03-06
  ref <308>: DATABASE ACCESSION NUMBER: GenBank/AF032099
  ref <309>: DATABASE ENTRY DATE: 1998-04-04
atgactacaaggaaagctcaagagatccacggaaaagcgccgggtggcagtgtttccacaggtgtgggaacggcagagggcgcccctagccccgcgggggtaacaccgccccctcccccgcgccctggccggactttccatgctatttttacccgccgacaccggacacccgactggggtggctgcggcgtcggggccacacgtccgttcaccggtcgcccgggctgtgcgcgccatggagccacggtgcccgccgccctgcgctgctgcgagcggctggtgctcaacgtggccgggttgcgcttcgagacccgcgcgcgcacgctcggccgcttcccggacacgctgctgggggacccggtgcgccgcagccgcttctacgacggcgcgcgcgccgagtatttcttcgaccgacaccggcccagcttcgatgcggtgctctactactaccagtcgggcggccggctgagacggccggcgcacgtgcccctcgacgtcttcctggaggaggtgtccttctacgggctggggcggcggctggcgcggctgcgggaggacgagggctgcgcggtcgccgagcggccgctgcccccgccctttgcgcgtcagctctggctgctcttcgaatttcctgagagctcgcaggctgcgcgcgtgctcgccgtggtctccgtactcgtcatcctggtctccatcgtggtcttttgcctcgagacactgccagacttccgcgacgaccgcgatgacccggggctcgcgccggtagcggctgctactggctcgttcctcgcccgactgaatggctccagtcccatgccaggagcccctccccgacagcccttcaacgatccattctttgtggtggagaccctgtgtatctgctggttctcctttgagctgctggtgcatctggtggcctgccctagcaaagctgtgttcttcaagaatgtgatgaacctaattgacttcgtggccatcctgccttacttcgtggccctgggcacggagttagcccggcagcggggtgtgggccagccggctatgtccctggccatcctaagggtcatccgattggtgcgtgtcttccgcatcttcaagctctccaggcattcgaagggtctacagatcttgggtcagacactgcgggcttccatgcgtgagctaggtctcctcatcttcttcctcttcattggcgtggtcctcttttccagcgcagtctactttgctgaagtggaccgggtggacacccatttcaccagcatcccggagtccttttggtgggcagtggtcaccatgaccacggttggctatggggacatggcacccgtcaccgtgggtggcaagatcgtgggctctctgtgtgccattgcaggtgtgctcaccatctctctgcctgtgcctgtcattgtctctaactttagctacttttaccaccgggagacagagggcgaagaggcagggatgtacagccatgtggacacacagccctgcggtaccctggagggcaaggctaatggggggctggtggactctgaggtgcctgaactcctcccaccactctggccccctgcagggaaacacatggtgactgaggtgtga

SEQ ID NO: 5 - DNA, 3473 bp, Mus musculus
  CDS (262)..(1860)
  ref <308>: DATABASE ACCESSION NUMBER: GenBank/NM_010596
  ref <309>: DATABASE ENTRY DATE: 2000-01-25
ctgcagatagtctcgggtggctcatgactgtcacccgggcaatccgaagatgaggcagaggattgccacaagttcgcggtcagcttaaatgacaggctgaggagactgtctaaataacaaacaaacaaataaatagccatgatgccatacgcctgcaatcccagcatttgggaggtggaggcaggaggatcataaattcaaggtcagttcagctacataagacctcgggtcatgaatccaaaagccaaacaaataaaatagatgactacaaggaaagctcaagagatccacggaaaagcgccgggtggcagtgtttccacaggtgtgggaacggcagagggcgcccctagccccgcgggggtaacaccgccccctcccccgcgccctggccggactttccatgctatttttacccgccgacaccggacacccgactggggtggctgcggcgtcggggccacacgtccgttcaccggtcgcccgggctgtgcgcgccatggagccacggtgcccgccgccctgcgctgctgcgagcggctggtgctcaacgtggccgggttgcgcttcgagacccgcgcgcgcacgctcggccgcttcccggacacgctgctgggggacccggtgcgccgcagccgcttctacgacggcgcgcgcgccgagtatttcttcgaccgacaccggcccagcttcgatgcggtgctctactactaccagtcgggcggccggctgagacggccggcgcacgtgcccctcgacgtcttcctggaggaggtgtccttctacgggctggggcggcggctggcgcggctgcgggaggacgagggctgcgcggtcgccgagcggccgctgcccccgccctttgcgcgtcagctctggctgctcttcgaatttcctgagagctcgcaggctgcgcgcgtgctcgccgtggtctccgtactcgtcatcctggtctccatcgtggtcttttgcctcgagacactgccagacttccgcgacgaccgcgatgacccggggctcgcgccggtagcggctgctactggctcgttcctcgctcggctcaatggctccagtcccatgccaggagcccctccccgacagcccttcaacgatccattctttgtggtggagaccctgtgtatctgctggttctcctttgagctgctggtgcatctggtggcctgccctagcaaagctgtgttcttcaagaatgtgatgaacctaattgacttcgtggccatcctgccttacttcgtggccctgggcacggagttagcccggcagcggggtgtgggccagccggctatgtccctggccatcctaagggtcatccgattggtgcgtgtcttccgcatcttcaagctctccaggcattcgaagggtctacagatcttgggtcagacactgcgggcttccatgcgtgagctaggtctcctcatcttcttcctcttcattggcgtggtcctcttttccagcgcagtctactttgctgaagtggaccgggtggacacccatttcaccagcatcccggagtccttttggtgggcagtggtcaccatgaccacggttggctatggggacatggcacccgtcaccgtgggtggcaagatcgtgggctctctgtgtgccattgcaggtgtgctcaccatctctctgcctgtgcctgtcattgtctctaactttagctacttttaccaccgggagacagagggcgaagaggcagggatgtacagccatgtggacacacagccctgcggtaccctggagggcaaggctaatggggggctggtggactctgaggtgcctgaactcctcccaccactctggccccctgcagggaaacacatggtgactgaggtgtgagggtcaactggggtctccagggagcagtggggtgggagggaggagggaaggcaggtcaggtgctgggttaaggactaagatggtaacaaaatcttaatctgaaggcatgtcacatggtggcgggtacagattcctagggggaccttatgtgacaggaattgccaggatttgggttgtgtccagggctccccccattgattgcgcaatgtggtagagctgtgcaaatgtccaggggctctatgggtagcactgtaagagacttggccatagattgtgagtttcctaggtgttcgtggggtcccactgggggcaagtgtggtcccgttaacacttgataggtactaatagaactttgggtcctctagggccaagtcggctcctgtaggtctctggagcactgttaaagagtgtgagcttgtggctacatggtcacacacagaggttatgtggtgatacatagcagattgtgtggattcatgtttcttctaggactcaagttattttagattctgtgagctctggagtcatgcagacaggatgtgatcactctgggtcctgatggaaaaagggttagggtataaaagatagacagaggcagagactcagataaagggacagagacacccccacacacacacacaacacacaattctctgtgttgtgcagggttataggactggaatcattttagggccatgcaaagctgagatggtgacccccatgtgtctccgaacagttgagtctgatgacttctacggtctcatgggacttgtgttactgtgagtcctgtggaacttttggttgaatttgactgtatgtgagggccattttaaacagtagatcagggtcacagcacagataaaactaactacaagggagcaagcatgggcacgtggatgtgtgtggcccgttgaacaagtttactcagggttatgcattgtagagctggtagtgtctgtatctctgggtccccctgcccgaagcacctagaaagacgatgggaactagtaacctgcctgtcttgctcatgtgggacccatatttctatgttgtcccatgctgtagtacaaagattcagaggctggttggagccacaaggaaagaaaacacagtagggcaagagtaggtacttgggaaaagatgtggcaggggacagagatctagaagaaagaaagatggagagaaggagcaggaggagcaggggagggggaaagagggaagggaaggagggagagacagagcaggtaaagaggaagagatgtgggggggggggggagaaggacctggagcccaagggtcatcctctctattctctgagtcataacagactctaagctatggcgtcagcatcccttagagccaaggaaggtcaccacactcctggaggtgctctgtgtttcctaggacagaaacccaaggctgtagtgtgctgtgcttcctcaagttacccagcggtcatagaccaagctggtgccaggtggcctgcctgtgacacccccacaacttctggtgtgcaggtattgcctgaggccactggtcctgtgtcctttgtttatcccttaaaacagggtccctctttgtagtgttggctgtcctggaactcactacgtaga

SEQ ID NO: 6 - protein, 532 aa, Mus musculus
MTTRKAQEIHGKAPGGSVSTGVGTAEGAPSPAGVTPPPPPRPGRTFHAIFTRRHRTPDWGGCGVGATRPFTGRPGCARHGATVPAALRCCERLVLNVAGLRFETRARTLGRFPDTLLGDPVRRSRFYDGARAEYFFDRHRPSFDAVLYYYQSGGRLRRPAHVPLDVFLEEVSFYGLGRRLARLREDEGCAVAERPLPPPFARQLWLLFEFPESSQAARVLAVVSVLVILVSIVVFCLETLPDFRDDRDDPGLAPVAAATGSFLARLNGSSPMPGAPPRQPFNDPFFVVETLCICWFSFELLVHLVACPSKAVFFKNVMNLIDFVAILPYFVALGTELARQRGVGQPAMSLAILRVIRLVRVFRIFKLSRHSKGLQILGQTLRASMRELGLLIFFLFIGVVLFSSAVYFAEVDRVDTHFTSIPESFWWAVVTMTTVGYGDMAPVTVGGKIVGSLCAIAGVLTISLPVPVIVSNFSYFYHRETEGEEAGMYSHVDTQPCGTLEGKANGGLVDSEVPELLPPLWPPAGKHMVTEV

SEQ ID NO: 7 - DNA, 522 bp, Mus musculus
  ref <308>: DATABASE ACCESSION NUMBER: GenBank/AI322534
  ref <309>: DATABASE ENTRY DATE: 1998-12-23
gtttccacaggtgtgggaacggcagagggcgcccctagccccgcgggggtaacaccgccccctcccccgcgccctggccggactttccatgctatttttacccgccgacaccggacacccgactggggtggctgcggcgtcggggccacacgtccgttcaccggtcgcccgggctgtgcgcgccatggagccacggtgcccgccgccctgcggctgctgcgagcggctggtgctcaacgtggccgggttgcgcttcgagacccgcgcgcgcacgctcggccgcttcccggacacgctgctgggggacccggtgcgccgcagccgcttctacgacggcgcgcgccgcgagtatttcttcgaccgacaccggcccagcttcgatgcggtgctctactactaccagtcgggcgggcggctgagaccggcggcgcaccttggccctcgacggtcttcctggaggaggtgtccttctacgggctgggcgcggcggcgcttgcgcggctgcgggaggacgagggctgc

SEQ ID NO: 8 - DNA, 468 bp, Mus musculus
  ref <308>: DATABASE ACCESSION NUMBER: GenBank/AI324179
  ref <309>: DATABASE ENTRY DATE: 1998-12-23
tcctcccgcagccgcgccagcgccgccgcgcccagcccgtagaaggacacctcctccaggaagacgtcgaggggcaggtgcgccggccgtctcagccggccgcccgactggtagtagtagagcaccgcatcgaagctgggccggtgtcggtcgaagaaatactcgcggcgcgcgccgtcgtagaagcggctgcggcgcaccgggtcccccagcagcgtgtccgggaagcggccgagcgtgcgcgcgcgggtctcgaagcgcaacccggccacgttgagcaccagccgctcgcagcagccgcagggcggcgggcaccgtggctccatggcgcgcacagcccgggcgaccggtgaacggacgtgtggccccgacgccgcagccaccccagtcgggtgtccggtgtcggcgggtaaaaatagcatggaaagtccggccagggcgcggtggagggggcggtgttacccccgc

SEQ ID NO: 9 - DNA, 27 bp, Artificial Sequence
  PCR primer
aaagctcaagagatccacggaaaagcg

SEQ ID NO: 10 - DNA, 27 bp, Artificial Sequence
  PCR primer
cgactggtagtagtagagcaccgcatc

SEQ ID NO: 11 - DNA, 3474 bp, Mus musculus
  CDS (497)..(1861)
ctgcagatagtctcgggtggctcatgactgtcacccgggcaatccgaagatgaggcagaggattgccacaagttcgcggtcagcttaaatgacaggctgaggagactgtctaaataacaaacaaacaaataaatagccatgatgccatacgcctgcaatcccagcatttgggaggtggaggcaggaggatcataaattcaaggtcagttcagctacataagacctcgggtcatgaatccaaaagccaaacaaataaaatagatgactacaaggaaagctcaagagatccacggaaaagcgccgggtggcagtgtttccacaggtgtgggaacggcagagggcgcccctagccccgcgggggtaacaccgccccctcccccgcgccctggccggactttccatgctatttttacccgccgacaccggacacccgactggggtggctgcggcgtcggggccacacgtccgttcaccggtcgcccgggctgtgcgcgccatggagccacggtgcccgccgccctgcggctgctgcgagcggctggtgctcaacgtggccgggttgcgcttcgagacccgcgcgcgcacgctcggccgcttcccggacacgctgctgggggacccggtgcgccgcagccgcttctacgacggcgcgcgcgccgagtatttcttcgaccgacaccggcccagcttcgatgcggtgctctactactaccagtcgggcggccggctgagacggccggcgcacgtgcccctcgacgtcttcctggaggaggtgtccttctacgggctggggcggcggctggcgcggctgcgggaggacgagggctgcgcggtcgccgagcggccgctgcccccgccctttgcgcgtcagctctggctgctcttcgaatttcctgagagctcgcaggctgcgcgcgtgctcgccgtggtctccgtactcgtcatcctggtctccatcgtggtcttttgcctcgagacactgccagacttccgcgacgaccgcgatgacccggggctcgcgccggtagcggctgctactggctcgttcctcgctcggctcaatggctccagtcccatgccaggagcccctccccgacagcccttcaacgatccattctttgtggtggagaccctgtgtatctgctggttctcctttgagctgctggtgcatctggtggcctgccctagcaaagctgtgttcttcaagaatgtgatgaacctaattgacttcgtggccatcctgccttacttcgtggccctgggcacggagttagcccggcagcggggtgtgggccagccggctatgtccctggccatcctaagggtcatccgattggtgcgtgtcttccgcatcttcaagctctccaggcattcgaagggtctacagatcttgggtcagacactgcgggcttccatgcgtgagctaggtctcctcatcttcttcctcttcattggcgtggtcctcttttccagcgcagtctactttgctgaagtggaccgggtggacacccatttcaccagcatcccggagtccttttggtgggcagtggtcaccatgaccacggttggctatggggacatggcacccgtcaccgtgggtggcaagatcgtgggctctctgtgtgccattgcaggtgtgctcaccatctctctgcctgtgcctgtcattgtctctaactttagctacttttaccaccgggagacagagggcgaagaggcagggatgtacagccatgtggacacacagccctgcggtaccctggagggcaaggctaatggggggctggtggactctgaggtgcctgaactcctcccaccactctggccccctgcagggaaacacatggtgactgaggtgtgagggtcaactggggtctccagggagcagtggggtgggagggaggagggaaggcaggtcaggtgctgggttaaggactaagatggtaacaaaatcttaatctgaaggcatgtcacatggtggcgggtacagattcctagggggaccttatgtgacaggaattgccaggatttgggttgtgtccagggctccccccattgattgcgcaatgtggtagagctgtgcaaatgtccaggggctctatgggtagcactgtaagagacttggccatagattgtgagtttcctaggtgttcgtggggtcccactgggggcaagtgtggtcccgttaacacttgataggtactaatagaactttgggtcctctagggccaagtcggctcctgtaggtctctggagcactgttaaagagtgtgagcttgtggctacatggtcacacacagaggttatgtggtgatacatagcagattgtgtggattcatgtttcttctaggactcaagttattttagattctgtgagctctggagtcatgcagacaggatgtgatcactctgggtcctgatggaaaaagggttagggtataaaagatagacagaggcagagactcagataaagggacagagacacccccacacacacacacaacacacaattctctgtgttgtgcagggttataggactggaatcattttagggccatgcaaagctgagatggtgacccccatgtgtctccgaacagttgagtctgatgacttctacggtctcatgggacttgtgttactgtgagtcctgtggaacttttggttgaatttgactgtatgtgagggccattttaaacagtagatcagggtcacagcacagataaaactaactacaagggagcaagcatgggcacgtggatgtgtgtggcccgttgaacaagtttactcagggttatgcattgtagagctggtagtgtctgtatctctgggtccccctgcccgaagcacctagaaagacgatgggaactagtaacctgcctgtcttgctcatgtgggacccatatttctatgttgtcccatgctgtagtacaaagattcagaggctggttggagccacaaggaaagaaaacacagtagggcaagagtaggtacttgggaaaagatgtggcaggggacagagatctagaagaaagaaagatggagagaaggagcaggaggagcaggggagggggaaagagggaagggaaggagggagagacagagcaggtaaagaggaagagatgtgggggggggggggagaaggacctggagcccaagggtcatcctctctattctctgagtcataacagactctaagctatggcgtcagcatcccttagagccaaggaaggtcaccacactcctggaggtgctctgtgtttcctaggacagaaacccaaggctgtagtgtgctgtgcttcctcaagttacccagcggtcatagaccaagctggtgccaggtggcctgcctgtgacacccccacaacttctggtgtgcaggtattgcctgaggccactggtcctgtgtcctttgtttatcccttaaaacagggtccctctttgtagtgttggctgtcctggaactcactacgtaga

SEQ ID NO: 12 - protein, 454 aa, Mus musculus
MEPRCPPPCGCCERLVLNVAGLRFETRARTLGRFPDTLLGDPVRRSRFYDGARAEYFFDRHRPSFDAVLYYYQSGGRLRRPAHVPLDVFLEEVSFYGLGRRLARLREDEGCAVAERPLPPPFARQLWLLFEFPESSQAARVLAVVSVLVILVSIVVFCLETLPDFRDDRDDPGLAPVAAATGSFLARLNGSSPMPGAPPRQPFNDPFFVVETLCICWFSFELLVHLVACPSKAVFFKNVMNLIDFVAILPYFVALGTELARQRGVGQPAMSLAILRVIRLVRVFRIFKLSRHSKGLQILGQTLRASMRELGLLIFFLFIGVVLFSSAVYFAEVDRVDTHFTSIPESFWWAVVTMTTVGYGDMAPVTVGGKIVGSLCAIAGVLTISLPVPVIVSNFSYFYHRETEGEEAGMYSHVDTQPCGTLEGKANGGLVDSEVPELLPPLWPPAGKHMVTEV

SEQ ID NO: 13 - DNA, 1668 bp, human
  promoter (1288)..(1567)
  misc_feature (1666)..(1668): ORF start
  misc_feature (1310)..(1310): Potential transcription start
  misc_feature (102)..(246): Regulatory region
aaaagaagagagaaggaatggaaggaaggaagacaggaagagaaagagtgccaggattagcagggcgttcgaacccttagtacctacctctctccacacccccagcagagggcgaccaggacttctttcctgggagtctcagtgatcaaacccctccacttcccccaagcccatctgatggagctatttttatgcagtgtcaccggacaccagtggctagaaggccaccgtcttgtctctgcgcccttcagttcctgaaaatgaccctgtggcatctccccggcccacacccccaacccccgcccgttccggcaagaccagactccttttgccagagtcatagccgcagctcaggtcctgctaggtctcagaaagtcttgcgaagaatccaggtggagactctggagcgaactctggggagagaatggggcgtgtgtgcacatgctcagtggtttgcagctgccagagtgaccctaggatgggacagccaccgggactgtggcagcacccacatggagtgatggtcagggagagatcaagagatatgactccggagaccctaagtcctctagagaccccaaggggagacccaaaggggagcggccgaggaaccctgagacagagacatggagggctaagagataatgagcaaaaagagacctagagaggagagacaaagagaccaagagagacaagagataaacggagacagaccaagagtcaaaataaaggcggagaccgagacgtagaggctggcagagggacccatacagaggcccccagaacgacgaatctcagaaataaatgacccatagagatactcagagatacatcccaaaagagacagagacccagaggggacagccggacacagacacacaagagagtgagagacaaaggtgcaaacagggcgactccaggccgggcgccgtggctcacgcttgtaaccccagcaattcgggaggccaaggagggcagatcacttgaggtcgggagtaccagacctgcctggccaacatggtgaaaccccttctcaactaaaaaagaaaaaagaaaaaaaattagctgggcttggtggcgcctgtaattccagctactagggaggctgaggcacgagaatcgcttgatcccgggaggtggaggttgttgcagtgagccgagatcgcgccactgcactccagcccgggcgacagagggaaactgtgtctcaaaaaaaaaaaaaaaaaaaaagacagaaagaaagaagaaagagaaaagaaaagaggcgactgcaactgaagcctgattctgacgaaacacacgcacacggaaacttggagagacgcaggacaggatcccggcggcagaaggacggagagaaaggggaccccgggacgggaaaggcgcagagcaggcgcgggcggcggcggcggcggggcagggcagggcgggcgtcccggcagagggcgcgcggtcgccctgtcgccctccgccccgccggggtcacagtgccccctccctcgcgccctagccgccctgccgggctatttttacgcgcggacaccggacaccggacaccgggctggggcggcggcggcggcggccgaggcggccgaggcggggccgcaccggggccgggcgtcggggccacacgtcggttcgcgggtcgccggggctgcgcgcgccatg

(19) United States
(12) Patent Application Publication
(10) Pub. No.: US 2002/0119476 A1
(43) Pub. Date: Aug. 29, 2002

(54) Nucleotide sequences coding for a mammalian coltage-gated potassium channel protein, the amino acid encoded by the nucleic acid and use thereof

(76) Inventors: Vladimir I. Kashuba, Spanga (SE); Claes Wahlestedt, Stockholm (SE); Eugene R. Zabarovsky, Upplands Vasby (SE)

Correspondence Address: DINSMORE & SHOHL, LLP, 1900 CHEMED CENTER, 255 EAST FIFTH STREET, CINCINNATI, OH, 45202, US

(21) Appl. No.: 09/993,811

(22) Filed: Nov. 16, 2001

Related U.S. Application Data
(60) Provisional application No. 60/253,548, filed on Nov. 28, 2000.
(60) Provisional application No. 60/275,327, filed on Mar. 13, 2001.

(30) Foreign Application Priority Data
Nov. 17, 2000 (SE) 0004228-3
Mar. 6, 2001 (SE) 0100742-6

(51) Int. Cl. C12Q 1/68; G01N 33/53; G01N 33/567; C07H 21/04; C12P 21/02; C12N 5/06; C07K 14/435

(52) U.S. Cl. 435/6; 435/7.2; 435/69.1; 435/325; 435/320.1; 530/350; 536/23.2

(57) ABSTRACT

The present invention relates to the mammalian KCNA7 gene coding for a voltage-gated potassium channel protein. The invention further relates to methods for identifying agents capable of modulating voltage-gated potassium ion channel activity. Blockers of the KCNA7 ion channel would be expected i.a. to increase insulin release and thereby reduce hyperglycemia associated with non-insulin-dependent diabetes mellitus

### TECHNICAL FIELD

The present invention relates to novel nucleic acid molecules coding for a mammalian voltage-gated potassium channel protein. The invention firer relates to methods for identifying agents capable of modulating voltage-gated potassium ion channel activity.

### BACKGROUND ART

Voltage-gated potassium ion channels (Kv channels), the largest sub-family of the ion channel superfamily, play important roles in a wide variety of cells (for reviews see Choe. S. et al. (1999) Trends Biochem. Sci. 24: 345-349; Lehmann-Hom, F. & Jurkat-Wottn K. (1999) Physiological Reviews 79; 1317-1372). From repolarizing membranes in response to action potentials, to regulating hormone secretion and calcium signaling, the potassium channels are critical genes for controlling the interactions of cells with their environments. Membrane depolarization activates voltage-gated potassium channels that, once opened, conduct potassium ions along the concentration gradient against the electric field. This outward current leads to repolarization of the membrane.

Kv channels in mammalian cells are encoded by an extended family of at least nineteen genes. The largest subfamily, Kv1, is related to the fly Shaker gene and contains at least seven members, Kv1.1-Kv1.7. The mammalian voltage-gated Shaker-related potassium-channel gene Kv1.7 (Kalman K. et al. (1998) J. Biol. Chem. 273; 5851-5857; see also U.S. Pat. No. 5.559,009) has been mapped to mouse chromosome 7 and human chromosome 19q 13.3. a region that has been suggested to contain a diabetic susceptibility locus.

Kv ion channels are in part responsible for the maintenance of cellular electrical activity. An imbalance in electrical activity is thought to be an underlying cause, at least in part, of several psychiatric diseases including schizophrenia, depression, anxiety, epilepsy, and neurodegenerative disorders (Herdon H. (1996) Potassium channel modulators and the central nervous system in Potassium channels and their modulators. Ed Evans J M. Hamilton T C, Longman S D, Stemp G. Taylor and Francis, Inc. pp. 361-383.) Thus compounds which maintain the electrical activity of the cell, would be expected to alleviate the symptoms of such diseases.

Therefore, ion channels may be useful targets for discovering ligands or drugs to treat many diverse disorders and defects, including schizophrenia, depression, anxiety, attention deficit hyperactivity disorder, migraine, stroke, ischemia, and neurodegenerative disease such as Alzheimer's disease, Parkinson's disease, glaucoma and macular degeneration. In addition compounds which modulate ion channels can be used for the treatment of cardiovascular diseases including ischemia, congestive heart failure, arrhythmia high blood pressure and restenosis.

Roe et al. (1996) J. Biol. Chem. 271:32241, demonstrated a correlation between channel blockade with the non-specific blockers tetraethylammonium and 4-aminopyridine and an increase in insulin release. Since voltage-gated potassium channels modulate insulin secretion from pancreatic β-cells, selective blockers of the new potassium ion channel would be expected to increase insulin release and thereby reduce hyperglycemia associated with non-insulin-dependent diabetes mellitus

An ongoing effort to create a physical framework for the human genome using NotI restriction sites has generated a large number of sequences preferentially containing exons (Kashuba V. I. et al. (1999) Gene 239: 259-271; Zabarovsky, E. R. et al. (2000) Nucleic Acids Research 239: 259-271). As NotI sites are preferentially detected in CpG (methylation-free) islands (Kashuba, V. I. et al., supra), and as most CpG islands include the 5-end of genes (Bird, A. (1987) Trends in Genetics 3(12): 342-347), the identification of sequences flanking NotI sites can aid in the identification of the edges of genes. This preference for regions adjoining or within genes has resulted in the discovery of a number of new genes.

### BRIEF DESCRIPTION OF THE DRAWINGS

FIG. 1

The KCNA7 gene. (A) Genomic structure of KCNA7, including a putative promoter and the exon positions. (B) Computational detection of potential skeletal muscle regulatory regions (boxes marked “Unknown TF”, cf. positions 105 to 114 and 201 to 211 in SEQ ID NO:13) in human KCNA7 gene and putative mouse sequence. Potential Sp1 and Mef2 binding sites are also shown (cf. positions 151 to 159 and 183 to 192 in SEQ ID NO:13).

FIG. 2 Alignment of the deduced amino acid sequence of KCNA7 with murine Kcna7. Black and gray boxes indicate identical and similar amino acid residues, respectively. (A) Alignment of N-termini of the human KCNA7 and murine Kcna7 (accession numbers AF032099 and NM_{—}010596.1). (B) Alignment of the human KCNA7 and murine Kcna7 proteins suggested after the introduction of an additional nucleotide G in the position 362 of the Kcna7 sequence (Accession no. NM_{—}010596.1).

FIG. 3 Hybridization of the KCNA7 to a Human Multiple Tissue Northern blot

### DESCRIPTION OF THE INVENTION

The present invention is directed to a novel, putative member of the mammalian voltage-gated potassium channel protein family, the potassium channel KCNA7. We report the cloning of the human ortholog to the murine Kv1.7 potassium ion channel, the tissue distribution of its expression, and the analysis of the genomic sequence encoding the gene.

The maximal open reading frame in the human gene encodes a protein of 456 amino acids (SEQ ID NO:2). The predicted product exhibits 91% amino acid identity to the murine voltage-gated potassium channel Kv1.7 (Kcna7SEQ ID NO; 6), which plays an important role in the repolarization of cell membranes. Based on the high similarity, the human protein has been classified as the ortholog of the mouse gene and designated KCNA7. A structural prediction identified a pore region characteristic of potassium channels and a transmembrane segment of the cyclic nucleotide gated channel. Northern expression analysis revealed the gene is expressed preferentially in skeletal muscle and heart. A single mRNA isoform was observed, with a size of approximately 4 kb. Using fluorescence in situ hybridization, the gene was mapped to chromosomal band 19q13.3. A genomic c sequence was identified in the database from this region, and the KCNA7 gene structure determined. Computational analysis of the genomic sequence reveals the location of a putative promoter and a likely muscle-specific regulatory region.

Further, a murine kcna7 gene sequence has been identified, which is different from the previously published murine sequence.

Consequently, in a first aspect this invention provides an isolated nucleic acid molecule selected from:

(a) nucleic acid molecules comprising a nucleotide sequence as shown in SEQ ID NO:1 or 11,

(b) nucleic acid molecules comprising a nucleotide sequence capable of hybridizing, under stringent hybridization conditions, to a nucleotide sequence complementary the polypeptide coding region of a nucleic acid molecule as defined in (a) and which codes for a biologically active KCNA7 polypeptide or a functionally equivalent modified form thereof; and

(c) nucleic acid molecules comprising a nucleic acid sequence which is degenerate as a result of the genetic code to a nucleotide sequence as defined in (a) or (b) and which codes for a KCNA7 polypeptide or a functionally equivalent modified form thereof.

The term “stringent hybridization conditions” is known in the art from standard protocols (e. Ausubel et at., supra) and could be understood as eg hybridization to filter-bound DNA in 0 5 M NaHPO₄, 7% sodium dodecyl sulfate (SDS), 1 mM EDTA at +65° C. and washing in 0.1×SSC/0.1 % SDS at +68° C.

The nucleic acid molecules according to the present invention includes cDNA, chemically synthesized DNA, DNA isolated by PCR, genomic DNA, and combinations thereof RNA transcribed from DNA is also encompassed by the present invention.

In a preferred form of the invention, the said nucleic acid molecule has a nucleotide sequence identical with SEQ ID NOS: 1 or 11 of the Sequence Listing. However, the nucleic acid molecule according to the invention is not to be limited strictly to the sequence shown as SEQ ID NO:1 or 11. Rather the invention encompasses nucleic acid molecules carrying modifications like substitutions, small deletions, insertions or inversions, which nevertheless encode proteins having substantially the biochemical activity of the mammalian KCNA7 polypeptide according to the invention. Included in the invention are consequently nucleic acid molecules, the nucleotide sequence of which is at least 90% homologous, preferably at least 95% homologous, with the nucleotide sequence shown as SEQ ID NO:1 or 11 in the Sequence Listing.

Included in the invention is also a nucleic acid molecule which nucleotide sequence is degenerate because of the genetic code, to the nucleotide sequence shown as SEQ ID NO:1 or 11. A sequential grouping of three nucleotides, a “codon”, codes for one amino acid Since there are 64 possible codons, but only 20 natural amino acids, most amino no acids are coded for by more than one codon. This natural “degeneracy”, or “redundancy”, of the genetic code is well known in the art. It will thus be appreciated that the nucleotide sequence shown in the Sequence Listing is only an example within a large but definite group of sequences which will encode the mammalian KCNA7 polypeptide

The invention also provides an isolated polypeptide encoded by the nucleic acid according to claim 1. In a preferred form, the said polypeptide has an amino acid sequence according to SEQ ID NO:2 or 12 of the Sequence Listing. However, the polypeptide according to the invention is not to be limited strictly to a polypeptide with an amino acid sequence identical with SEQ ID NO:2 or 12 in the Sequence Listing. Rather the invention encompasses polypeptides carrying modifications like substitutions, small deletions, insertions or inversions, which polypeptides nevertheless have substantially the biological activities of mammalian KCNA7.

In another aspect, the invention provides a vector harboring the nucleic acid molecule as defined above. The term “vector” refers to any carrier of exogenous DNA that is useful for transferring the DNA to a host cell for replication and/or appropriate expression of the exogenous DNA by the host cell. The said vector can e.g. be a replicable expression vector, which carries and is capable of mediating the expression of a DNA molecule according to the invention. In the present context the term “replicable” means that the vector is able to replicate in a given type of host cell into which is has been introduced. Examples of vectors are viruses such as bacteriophages, cosmids, plasmids and other recombination vectors. Nucleic acid molecules are inserted into vector genomes by methods well known in the art.

Included in the invention is also a cultured host cell harboring a vector according to the invention. Such a host cell can be a prokaryotic cell, a unicellular eukaryotic cell or a cell derived from a multicellular organism The host cell can thus e.g. be a bacterial cell such as an E. Coli cell; a cell from yeast such as Saccharomyces cervisiac or Pichia pastoris, or a mammalian cell. The methods employed to effect introduction of the vector into the host cell are standard methods well known to a person familiar with recombinant DNA methods. A further aspect of the invention is a process for production of a mammalian KCNA7 polypeptide which comprises culturing a host cell as defined above under conditions whereby said polypeptide is produced, and recovering said polypeptide.

In yet an important aspect, this invention provides a method for identifying an agent capable of modulating voltage-gated potassium ion channel activity, comprising

(i) providing a cell expressing the mammalian KCNA7 polypeptide;

(ii) contacting said cell with a candidate agent; and

(iii) monitoring said cell for an effect that is not present in the absence of said candidate agent As used herein, the term “agent” means a biological or chemical compound such as a simple or complex organic molecule, a peptide, a protein or an oligonucleotide.

Specifically, such a method can comprise the steps (i) contacting a candidate agent with a nucleic acid molecule according to the invention, or with the encoded mammalian KCNA7 polypeptide; and (ii) determining whether said candidate agent modulates the expression of the said nucleic acid molecule, or whether the candidate agent modulates the biological activities of the said polypeptide. In this context, the term “biological activities” is intended to encompass triggering release or uptake of protein or non-protein molecules from the cell, or triggering the opening of the channels and the movement of ions across the cellular membrane, and the electrical signal which accompanies the passage of the ions across the membrane as assessed with the technique of electrophysiology. For example, activity can be determined by measuring ion flux. As used herein, the term “ion flux” includes ion current. Activity can also be measured by measuring changes ink membrane potential using electrodes or voltage-sensitive dyes, or by measuring neuronal or cellular activity such as action potential duration or frequency, the threshold for stimulating action potentials, long-term potentiation or long-term inhibition. For references, see e.g. “Electroplhysiology, A Practical Approach”, D I Wallis (ed.), IRL Press at Oxford University Press, 1993; or “Voltage and patch Clamping wit Microelectrodes”, T G Smith, H Lecar, S T Redman and P W Gage (eds), Waverly Press, Inc for the American Physiology Society, 1985.

Blockers of the mammalian KCNA7 ion channel would be expected to increase insulin release and thereby reduce hyperglycemia associated with non-insulin-dependent diabetes mellitus. Consequently, agents modulating the mammalian KCNA7 gene or KCNA7 protein could be useful for the treatment of diabetes and related medical conditions.

Further, modulators of the mammalian KCNA7 ion channel could be useful in the treatment of ion channel related problems such as schizophrenia, depression, anxiety, attention deficit hyperactivity disorder migraine, stroke, ischemia, glaucoma, macular degeneration, epilepsy, and neurodegenerative disease such as Alzheimer's disease and Parkinson's disease.

For screening purposes, appropriate host cells can be transformed with a vector having a reporter gene under the control of the KCNA7 gene according to this invention. The expression of the reporter gene can be measured in the presence or absence of an agent with known activity (i.e. a standard agent) or putative activity (i.e. a “test agent” or “candidate agent”). A change in the level of expression of the reporter gene in the presence of the candidate agent is compared with that effected by the standard agent. In this way, active agents are identified and their relative potency in this assay determined.

As used herein, the term “reporter gene” means a gene encoding a gene product that can be identified using simple, inexpensive methods or reagents and that can be operably linked to the KCNA7 gene or an active fragment thereof Reporter genes such as, for example, a luciferase, β-galactosidase, alkaline phosphatase, or green fluorescent protein reporter gene, can be used to determine transcriptional activity in screening assays according to the invention (see, for example, Goeddel (ed.), Methods Enzymol., Vol. 185, San Diego: Academic Press, Inc. (1990); see also Sambrook, supra).

The effect of candidate agents on the KCNA7 potassium channel can be monitored by methods known in the art. For instance, the rate of ⁸⁶Rb efflux from a ⁸⁶Rb loaded cell, expressing the mammalian KCNA7 ion channel, can be monitored (cf. Example 7, below). When the candidate agent is a polypeptide, its interaction with the KCNA7 polypeptide cat be monitored by well known methods for determination of protein-protein interactions. Examples of such methods, applicable for the soluble portion of KCNA7, are the yeast two-hybrid system and FRET (fluorescence resonance energy transfer) (cf. Examples 8 and 9, respectively). Another example is determination of changes in membrane potential using the FLIPR system (cf. Example 10)

In a further aspect, the invention provides a method for the identification of an agent modulating transcription of the human KCNA7 gene, said method comprising the steps

(i) contacting a candidate agent with a regulatory region shown as positions 102 to 246, or a part thereof, such as in particular positions105 to 114 or 201 to 211, in SEQ ID NO: 13. and

(ii) determining whether said candidate agent modulates expression of the human KCNA7 gene, such modulation being indicative for an agent modulating transcription of the human KCNA7 gene.

In a particular embodiment, the novel molecules identified by the screening methods according to the invention are low molecular weight organic molecules, in which case a composition or pharmaceutical composition can be prepared thereof for oral intake, such as in tablets. The compositions, or pharmaceutical compositions, comprising the nucleic acid molecules, vectors, polypeptides, antibodies and compounds identified by the screening methods described herein, can be prepared for any route of administration including but not limited to, oral, intravenous, cutaneous, subcutaneous, nasal, intramuscular or intraperitoneal. The nature of the carrier or other ingredients will depend on the specific route of administration and particular embodiment of the invention to be administered. Examples of techniques and protocols that are useful in this context air, inter alia, found in Remington's Pharmaceutical Sciences, 16^{th} edition, Osol, A. (ed.). 1980, which is incorporated herein by reference in its entirety.

The dosage of these low molecular weight compounds will depend on the disease state or condition to be treated and other clinical factors such as weight and condition of the human or animal and the route of administration of the compound. For treating human or animals, between approximately 0.5 mg/kg of body weight to 500 mg/kg of body weight of the compound can be administered. Therapy is typically administered at lower dosages and is continued until the desired therapeutic outcome is observed.

### Materials and Methods

cDNA library from heart (Stratagene, La Jolla, Calif., USA) in λ ZAP II was used for the screening and isolation of cDNA clones. Marathon-Ready™ cDNA from skeletal muscle (Clontech, Palo Alto, Calif., USA) was used for 5′- and 3′-RACE PCR.

Homology searches were performed using BLASTX and BLASFN programs (Altscul et al. (1997) Nucleic Acids Res. 25:3389-3402; Gish & States (1993) Nature Genetics 3: 266-272) Software for performing BLAST analyses is publicly available through the National Center for Biotechnology Information (http://wwwncbi.nlm.nih.gov). A search for motifs common to protein families was performed with the Prosite motif library. Information about the Prosite motifs is available at the ISREC server (http//www.isrec.isb-sib.ch).

A prediction of membrane-spanning regions and their orientation was made. Software for the prediction of transmembrane regions is available through various sources, e.g. at http.//www ch. embnet org/software/TMPRED_form.html. Exon positions in the genomic sequence were determined with “est_genome” (http://www.sanger ac.uk), a specialized tool for the prediction of exon boundaries using ESTs (See Mott. R. (1997) Computer Applications in the Biosciences 13(4): 477-478). Promoter prediction was performed on the genomic sequence with the algorithm “PromoterInspector” (Scherf, M. et al. (2000) J. Mol. Biol. 297: 599-606). The positions of putative transcription regulatory regions for muscle-specific expression were determined with a logistic regression model (Wasserman, W. W. & Fickett, J. W. (1998) J. Mol. Biol. 278; 167-181).

Throughout this description the terms “standard methods” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook. J., Fritsch, E. F. and Maniatis, T., Molecular Cloning: A laboratory manual, 2nd Ed., Cold Spring Harbor Laboratory Press. Cold Spring Harbor, N.Y. 1989.

Additional features of the invention will be apparent from the following Examples. Examples 1 to 5 are actual, while Examples 6 to 10 are prophetic.

### EXAMPLES OF THE INVENTION

### EXAMPLE 1

### Identification of Human KCNA7

Construction of NotI linking libraries was described previously (Zabarovsky et al. (1994) Genomics 20: 312-316; Zabarovsky et al. (2000) Nucleic Acids Research 28: 1635-1639). The No/I linking clone NR1-253 (SEQ ID NO:3; GenBank Accession No. AQ939522. approximately 900 bp) displayed 85% identity over 658 nucleotides with the murine voltage-gated potassium channel Kv1.7 (Kcna7) (SEQ ID NO:4; GenBank Accession No. AF032099; Kalman, K. et al. (1998) J. Biol. Chem. 273: 5851-5857).

A set of overlapping human cDNA sequences was obtained via a combination of cDNA library, screening and RACE-PCR according to standard methods. The final full-length human sequence (4372 bp, SEQ ID NO:1) was obtained by the fusion of the longest 3′-and 5′-RACE PCR products with cDNA clone (p5kv1-7) containing the complete ORF. The KCNA7 nucleotide sequence encodes a deduced protein of 456 amino acids (SEQ ID NO:2)

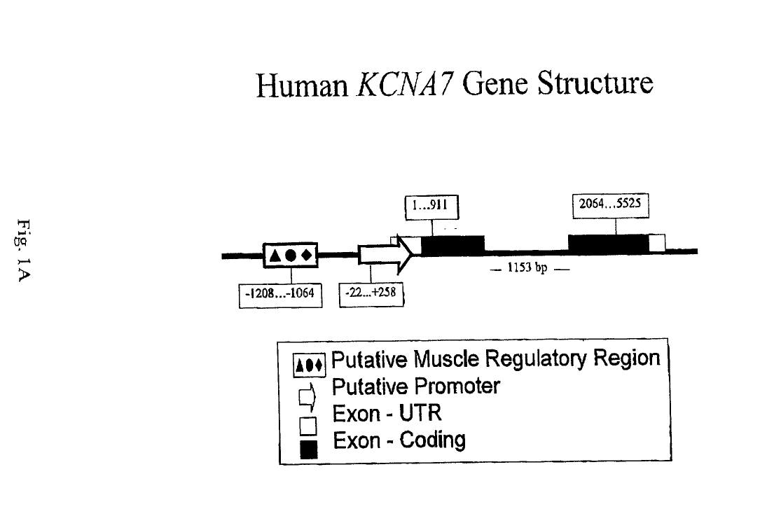

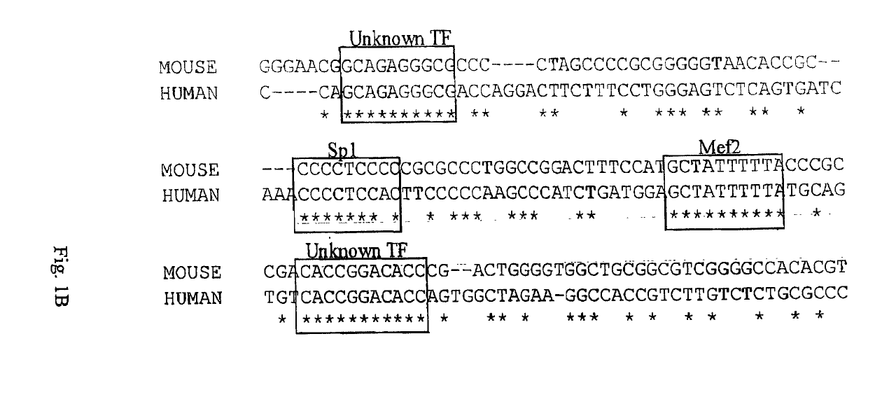

Screening the genome sequence database revealed the KCNA7 gene in the draft sequence of the human BAC clone CTB-60B 18 (GenBank accession number AC008687.3; positions 84249-83339 and 82186-78726; reverse orientation). Like the murine ortholog, but unlike other human Kv1 channels genes, the human KCNA7 gene is split into taco exons of length 911 and 3461 bp, respectively (FIG. 1A; positions 1-911 and 912-4372 in SEQ ID NO 1). The human gene intron is 1153 bp in length, compared to the reported murine intron of length 1.9 kb (Kalman et al., supra).

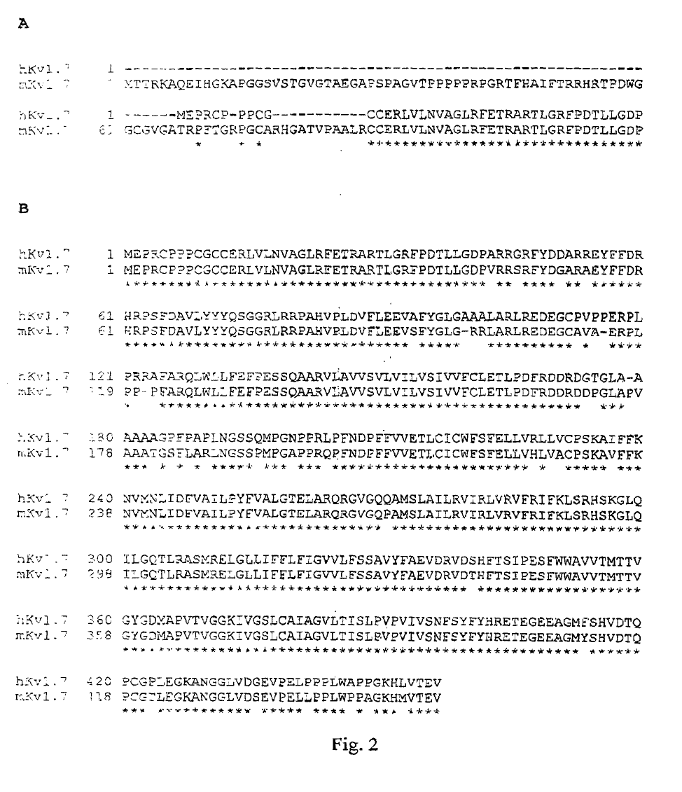

The human KCNA7 protein (SEQ ID NO:2) displays high level of amino acid identity to the mouse Kcna7 protein (SEQ ID NO:6) (90% in 452 aa overlap, score 809 bits) and less similarity to a variety of human potassium channel genes (<71% in 411-434 aa overlap). In fact, in many extended regions the human and mouse genes are identical (FIG. 2). Based on the observed similarity, we postulate that the human and murine genes are true orthologs. Nucleic acid similarity is less profound, in the best cases reaching 82%-86%, which is consistent with the variability between orthologous human and rodent genes (Makalowski, W. & Boguski, M. S. (1998) Proc. Natl. Acad. Sci. of U.S.A. 95(16): 9407-9412).

The deduced human KCNA7 protein contains six putative membrane-spanning domains. The region between amino acids 237 and 448 is recognized as a transmembrane part of the cyclic nucleotide gated channel and contains a pore region amino acids 342-397). The protein contains conserved sites for various post-translational modifications. As other Shaker-related channels, KCNA7 has a potential tyrosine kinase phosphorylation site (RPSFDAVLY) in its N-terminal region (amino acids 62-70) (Chandy, K. G. & Gutman, G. A (1995) in Handbook of Receptors and Channels: Ligand and Voltage-gated Ion Channels North, A. ed.) pp.1-72, CRC Press, Boca Raton, Fla.). Two protein kinase C consensus sites. viz. TLR (amino acids 304-306) and SMR (amino acids 308-310) are present in the cytoplasmic loop; at least one of these sites is present in all members of the Kv1 family (Chandy & Gutman, supra).

### Example 2

### Identification of a Frameshift in the Published Murine cDNA Sequence

The human and murine cDNA sequences indicate different N-terminal sequences in the encoded polypeptides (FIG. 2A). Potassium channels can vary significantly at the 5′-ends but as these orthologous genes are highly similar, it seems more likely that a frameshift within the murine cDNA sequence could have produced an inaccurate N-terminal sequence.

Consistent with this hypothesis, the human cDNA sequence contains the sequence CGGC at positions 383-386, which corresponds to the sequence CGC at positions 523-525 in the murine cDNA sequence (SEQ ID NO:5). In support of this difference being a frameshift error, the murine ESTs for Kcna7 (Accession numbers AI1322534.1 (SEQ ID NO; 7) and A1324179 (SEQ ID NO:8)) contain the sequence CGGC Further, a murine genomic sequence (GenBank accession no. AC073711) for the Kcna7 gene, or possibly for a recently created paralog or pseudogene, contains the CGGC sequence. If the CGGC sequence is correct, the murine ORF would be altered at the N-terminal such that the first 88 amino acids of the published murine sequence would be replaced by 10 amino acids identical to the human N-terminal sequence. With this correction to the murine sequence, it would show 91% identity with the human sequence (FIG. 2B).

To directly check the sequence of murine Kcna7 gene we performed PCR with mouse genomic DNA and the PCR primers shown as SEQ ID NOS: 9 and 10. Sequencing of the PCR product, according to standard procedures, confirmed that the mouse gene comprises the CGGC sequence and therefore its N-terminal protein sequence is identical to the human. The corrected murine nucleotide and amino acid sequences are shown as SEQ ID NOS; 11 and 12, respectively.

### Examples 3

### Expression Analysis

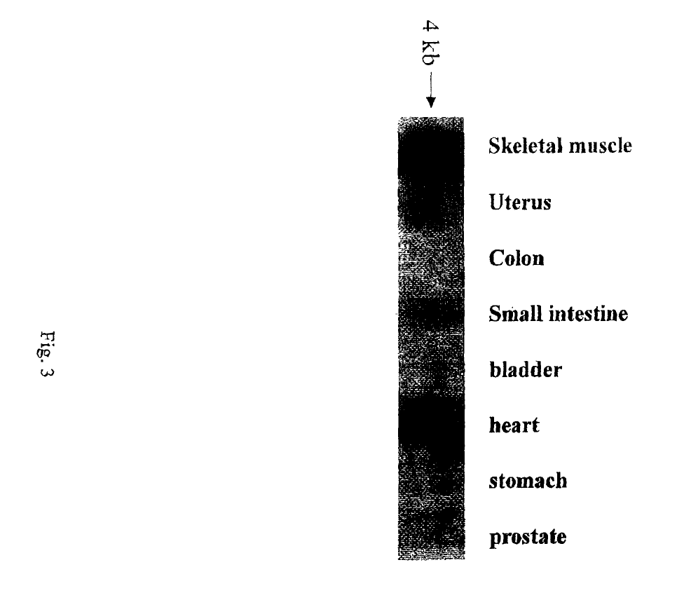

Northern hybridization of the cloned human gene was carried out using a filter containing RNA from a variety of muscle tissues (Clontech, Human Muscle #7765-1). Northern expression analysis revealed the highest expression of KCNA7 in skeletal muscle and heart (FIG. 3). A single band of approximately 4 kb was observed. Skeletal muscle is believed to be the principal tissue responding to insulin to modify glucose levels in the body (see e.g. Zierath, J. R. et al (2000) Diabetologia 43:821-835). Expression in smooth muscles was detected at a lower level consistent with the expression of murine Kcna7.

### Example 4

### Identification of Putative Regulatory Regions

Analysis of the human genomic sequence suggestions the location of some regulatory control regions The “PromoterInspector” algorithm (Scherf, M. et at. (2000) J. Mol Biol 297. 599-606) suggested the presence of a single promoter adjacent to the identified first exon. A unique algorithm (see Wasserman, W. W. & Ficket:, J. W. (1998) J. Mol. Biol. 278: 167-181) for the identification of transcriptional regulatory regions directing skeletal muscle-specific transcription was applied to the KCNA7 genomic sequence. A putative regulatory region was identified at approximately −1100 relative to the 5′-end of the first exon (FIG. 1B; cf. positions 102 to 246 in SEQ ID NO:13. Potential binding sites for both Mef-2 and Sp-1 transcription factors (Wasserman & Fickett.,supra) were identified within this region.

### Example 5

### In situ Hybridization

Fluorescence in situ hybridization (FISH) analysis with metaphase chromosomes was performed a, described previously (Protopopov et al. (1996) Chromosome Research 4:443-447). The NotI linking clone NR1-253, to which the KCNA7 gene corresponds (see Example 1, above), was assigned to chromosomal band 19q 13.3. As previously observed (Kalman, K. et al. (1998) J. Biol. Chem. 273: 5851-5857), this map location is consistent with a putative diabetes susceptibility gene that has been suggested to be present al 19q 13.3 This suggestion is especially strong for Finnish families wit associated hypertension and difficulties ill insulin-stimulated glucose storage (Groop, L. C. et al (1993) New Engl. J. Medicine 328: 10-14; Lehto, M. et al. (1993) Genomics 15: 460-461 Elbein S. C. et al. (1994) Diabetes 43: 1061-1065). For the Kcna7 gene, expression in mouse pancreatic islet cells was demonstrated (Kalman et al., supra). Thus, human KCNA7 could be linked to the pathogenesis of type II diabetes mellitus, in some humans.

### Example 6

### Expression of Voltage-gated Ion Channel Polypeptides in Mammalian Cells

(a) Expression of Voltage-gated Ion Channel Polypeptides in 293 Cells

For expression of voltage-gated ion channel polypeptides in mammalian cells 293 (transformed human, primary embryonic kidney cells), a plasmid bearing the relevant voltage-gated ion channel coding sequence is prepared, using vector pCDNA6 (Invitrogen) Vector pCDNA6 contains the CMV promoter and a blasticidin resistant gene for selection of stable transfectants. Many other vectors can be used containing, for example, different promoters, epitope tags for detection and/or purification of the protein, and resistance genes. The forward primer for amplification of this voltage-gated ion channel polypeptide encoding cDNA is determined by procedures as well known in the art and preferably contains a 5′ extension of nucleotides to introduce the HindIII cloning site and nucleotides matching the voltage-gated ion channel nucleotide sequence. The reverse primer preferably contains a 5′ extension of nucleotides to introduce an XhoI restriction site for cloning and nucleotides corresponding to the reverse complement of the voltage-gated ion channel nucleotide sequence. The PCR conditions are 5° C. as the annealing temperature. The PCR product is gel purified and cloned into the HindIII-XhoI sites of the vector.

The DNA is purified using Qiagen chromatography columns and transfected into 293 cells using DOTAP transfection media (Boehringer Mannheim, Indianapolis, Ind.). Transiently n-transfected cells are tested for expression after 24 hours of transfection, using Western blots probed with anti-His and anti-voltage-gated ion channel peptide antibodies Permanently transfected cells are selected with Zeocin and propagated. Production of the recombinant protein is detected from both cells and media by western blots probed with anti-His, anti-Myc or anti-voltage-gated ion channel peptide antibodies.

(b) Expression of Voltage-gated Ion channel Polypeptides in COS Cells

For expression of voltage-gated ion channel polypeptides in COS7 cells, a polynucleotide molecule having a nucleotide sequence of SEQ ID NO:1 or complementary nucleotide sequences thereof, can be cloned into vector p3-CI. This vector is a pUC18-derived plasmid that contains the HCMV (human cytomegalovirus) promoter-intron located upstream from the bGH (bovine growth hormone) polyadenylation sequence and a multiple cloning site. In addition, the plasmid contains the dhrf (dihydrofolate reductase) gene which provides selection in the presence of the drug methotrexane (MTX) for selection of stable transformants. Many other vectors can be used containing, for example, different promoters, epitope tags for detection and/or purification of the protein, and resistance genes.

The forward primer is determined by procedures known in the art and preferably contains a 5′ extension which introduces an XbaI restriction site for cloning, followed by nucleotides which correspond to a nucleotide sequence given in SEQ ID NO:1, or portion thereof The reverse primer is also determined by methods well known in the art and preferably contains a 5′-extension of nucleotides which introduces a SalI cloning site followed by nucleotides which correspond to the reverse complement of a nucleotide sequence given in SEQ ID NO:1, or portion thereof.

The PCR consists of an initial denaturation step of 5 min at 95° C., 30 cycles of 30 sec denaturation at 95° C., 30 sec annealing at 58° C. and 30 sec extension at 72°C., followed by 5 min extension at 72° C. The PCR product is gel purified and ligated into the XbaI and SalI sites of vector p3-CI. This construct is transformed into E. coli cells for amplification and DNA purification. The DNA is purified with Qiagen chromatography columns and transfected into COS 7 cells using Lipofectamine reagent (Gibco/BRL), following the manufacturer's protocols. Forty-eight and 72 hours after transfection, the media and the cells are tested for recombinant protein expression.

Voltage-gated ion channel polypeptides expressed in cultured COS cells can be purified by disrupting cells via homogenization and purifying membranes by centrifugation, solubilizing the protein using a suitable detergent, and pawing the protein by, for example, chromatography. Purified voltage-gated ion channel is concentrated to about 0.5 mg/ml in an Amicon concentrator fitted with a YM-10 membrane and stored at fan −80° C.

### Example 7

### Use of Kv1.7 Expression Construct to Identify Kv1.7-specific Glucose-dependent Insulin Secretagogues

The KCNA7 expression construct [described above] can be used to generate functional potassium channels with unique properties. This construct can be used for expression of functional KCNA7 channels in mammalian cell lines that do not express endogenous potassium channels (e.g. CV-1, NTH-3T3, or RBL cell lines). These cell lines can then be loaded with ⁸⁶Rb (Rb ions permeate through potassium channels nearly as well as potassium ions) in the presence of absence of extrinsic materials, and KCNA7 modifiers identified by their ability to alter ⁸⁶Rb-efflux. When natural toxins are identified which block KCNA7 activity, modifiers of KCNA7 activity could also be identified by their ability to block or reverse the binding of labeled toxins to cells expressing this channel. Compounds discovered in either of these manners could then be formulated and administered as therapeutic agents for the treatment of NIDDM.

### Example 8

### Interaction Trap/Two-Hybrid System

In order to assay for voltage-gated ion channel polypeptide-interacting proteins, the interaction trap/two-hybrid library screening method can be used. This assay was first described in Fields & Song (1989) Nature 340: 245-246. A protocol is published in Current Protocols in Molecular Biology 1999, John Wiley & Sons. New York, and Ausubel, F. M. et al. 1992. Short Protocols in Molecular Biology, 4^{th} ed., Greene and Wiley-Interscience, NY. Kits are available from Clontech, Palo Alto, Calif. (Matchmaker Two-Hybrid System 3).

A fusion of the nucleotide sequences encoding an intracellular, soluble portion of the voltage-gated ion channel polypeptide and the yeast transcription factor GAL4 DNA-binding domain (DNA-BD) is constructed in an appropriate plasmid (i.e., pGBKT7), using standard subcloning techniques. Similarly, a GAL4 active domain (AD) fusion library is constructed in a second plasmid (i.e., pGADT7) from cDNA of potential voltage-gated ion channel polypeptide-binding proteins. The DNA-BD/voltage-gated ion channel fusion construct is verified by sequencing, and tested for autonomous reporter gene activation and cell toxicity, both of which would prevent a successful two-hybrid analysis. Similar controls are performed with the AD/library fusion construct to ensure expression in host cells and lack of transcriptional activity. Yeast cells are transformed (ca. 10⁵ transformants/mg DNA) with both the voltage-gated ion channel and library fusion plasmids according to standard procedures. In vivo binding of DNA-BD/voltage-gated ion channel with AD/library proteins results in transcription of specific yeast plasmid reporter genes (i.e., lacZ, HIS3, ADE2, LEU2). Yeast cells are plated on nutrient-deficient media to screen for expression of reporter genes. Colonies are dually assayed for β-galactosidase activity upon growth in Xgal (5-bromo-4-chloro-1-indolyl-β-D-galactoside) supplemented media (filter assay for β-galactosidase activity is described in Breeden, et al., Cold Spring Harb. Symp. Quant. Biol., 1985, 50, 643). Positive AD-library plasmids are rescued from transformants and reintroduced into the original yeast strain as well as other strains containing unrelated DNA-BD fusion proteins to confirm specific voltage-gated ion channel polypeptide/library protein interactions. Insert DNA is sequenced to verify the presence of an open reading frame fused to GAL4 AD and to determine the identity of the voltage-gated ion channel polypeptide-binding protein.

### Example 9

### FRET Analysis of Protein-Protein Interactions

In order to assay for voltage-gated ion channel polypeptide-interacting proteins, fluorescence resonance energy transfer (FRET) methods can be used. An example of this type Of assay is described in Mahajan, N. P. et al. (1998) Nature Biotechnology 16: 547-552. This assay is based on the fact that when two fluorescent moieties having the appropriate excitation/emission properties are brought into close proximity, the donor fluorophore, when excited, can transfer its energy to the acceptor fluorophore whose emission is measured. The emission spectrum of the donor must overlap with the absorption spectrum of the acceptor while overlaps between the two absorption spectra and between the two emission spectra, respectively, should be minimized. An example of a useful donor/acceptor pair is Cyan Fluorescent Protein (CFP)/Yellow Fluorescent Protein (YFP) (Tsien (1998) Annu. Rev. Biochem. 67, 509-544).

A fusion of the nucleotide sequences encoding an intracellular soluble portion of the voltage-gated ion channel polypeptide and CFP is constructed in an appropriate plasmid, using standard subcloning techniques. Similarly, a nucleotide encoding a YFP fusion of the possibly interacting target protein is constructed in a second plasmid The CFP/voltage-gated ion channel polypeptide fusion construct is verified by sequencing. Similar controls are performed with the YFP/target protein construct The expression of each protein can be monitored using fluorescence techniques (e.g., fluorescence microscopy or fluorescence spectroscopy). Host cells are transformed with both the CFP/voltage-gated ion channel polypeptide and YFP/target protein fusion plasmids according to standard procedure. In situ interactions between CFP/voltage-gated ion channel polypeptide and the YFP/target protein are detected by monitoring the YFP fluorescence after exciting the CFP fluorophore. The fluorescence is monitored using fluorescence microscopy or fluorescence spectroscopy. In addition, changes in the interaction due to e.g., external stimuli are measured using time-resolved fluorescence techniques.

Alternatively, a YFP fusion library may be constructed from cDNA of potential voltage-gated ion channel polypeptide-binding proteins Host cells are transformed with both the CFP/voltage-gated ion channel polypeptide and YFP fusion library plasmids. Clones exhibiting FRET are then isolated and the protein interacting with a voltage-gated ion channel polypeptide is identified by rescuing and sequencing the DNA encoding the YFP/target fusion protein.

### Example 10

### High Throughput Screening for Modulators of Ion Channels Using FLIPR

One method to identify compounds that modulate the activity of an ion channel polypeptide is through the use of the FLIPR (Fluorometric Imaging Plate Reader) system, which is developed to perform cell-based, high-throughput screening (HTS) assay s measuring, for example, membrane potential (For a review, see Schroeder K. S. and Neagle B. D. (1996) FLIPR: a new instrument for accurate, high throughput optical screening. J. Biomol. Screen. 1: 75-80). Changes in plasma membrane potential correlate with the modulation of ion channels, as ions move into or out of the cell The FLIPR system measures such changes in membrane potential. This is accomplished by loading cells expressing an ion channel gene with a cell-membrane permeable fluorescent indicator dye suitable for measuring changes in membrane potential such as diBAC (bis-(1,3-dibutylbarbituric acid)pentamethine oxonol, Molecular Probes). Thus the modulation of ion channel activity is assessed with FLIPR and detected as changes in the emission spectrum of the diBAC dye.

As an example, COS cells that have been transfected with an ion channel gene of Interest are bathed in diBAC. Due to the presence of both endogenous potassium channels in the cells as well as the transfected channel, the addition of 30 mM extracellular potassium causes a membrane depolarization This results in an increase in diBAC uptake by the cell, and thus an overall increase in fluorescence. When cells ale treated with a potassium channel opener, such as chromakalim, the membrane is hyperpolarized causing a net outflow of diBAC, and thus a reduction in fluorescence. In this manner the effect of unknown test compounds on membrane potential can be assessed using this assay.

1. An isolated nucleic acid molecule selected from:
(a) nucleic acid molecules comprising a nucleotide sequence as shown in SEQ ID NO:1 or 11:
(b) nucleic acid molecules comprising a nucleotide sequence capable of hybridizing, under stringent hybridization conditions, to a nucleotide sequence complementary the polypeptide coding region of a nucleic acid molecule as defined in (a) and which codes for a biologically active KCNA7 polypeptide or a functionally equivalent modified form thereof; and
(c) nucleic acid molecules comprising a nucleic acid sequence which is degenerate as a result of the genetic code to a nucleotide sequence as defined in (a) or (b) and which codes for a KCNA7 polypeptide or a functionally equivalent modified form thereof.

2. An isolated mammalian KCNA7 polypeptide encoded by the nucleic acid according to claim 1.

3. An isolated human KCNA7 polypeptide having an amino acid sequence shown as SEQ ID NO:2 in the sequence listing.

4. An isolated murine kcna7 polypeptide having an amino acid sequence shown as SEQ ID NO:12 in the sequence listing.

5. A vector harboring the nucleic acid molecule according to claim 1.

6. A replicable expression vector, which carries and is capable of mediating the expression of a nucleic acid molecule according to claim 1.

7. A cultured host cell harboring a vector according to claim 5 or 6.

8. A process for production of a mammalian KCNA7 polypeptide which comprises culturing a host cell according to claim 7 under conditions whereby said polypeptide is produced, and recovering said polypeptide.

9. A method for identifying an agent modulating voltage-gated potassium ion channel activity, comprising
(i) providing a cell expressing the mammalian KCNA7 polypeptide according to any one of claims 2 to 4;
(ii) contacting said cell with a candidate agent; and
(iii) monitoring said cell for an effect Stat is not present in the absence of said candidate agent.

10. A method for identifying an agent modulating the expression of a mammalian KCNA7 nucleic acid molecule, said method comprising the steps
(i) contacting a candidate agent with a nucleic acid molecule according to claim 1; and
(ii) determining whether said candidate agent modulates the expression of the said nucleic acid molecule.

11. A method for identifying an agent useful for the treatment of diabetes, said method comprising the steps
(i) contacting a candidate agent with a nucleic acid molecule according to claim 1; and
(ii) determining whether said candidate agent decreases or inhibits the expression of the nucleic acid molecule, such decrease or inhibition being indicative for a compound useful for the treatment of diabetes.

12. A method for identifying an agent useful for the treatment of ion channel related conditions selected from the group consisting of schizophrenia, depression, anxiety, attention deficit hyperactivity disorder, migraine, stroke, ischemia, glaucoma, macular degeneration, epilepsy, and neurodegenerative disease, said method comprising the steps
(i) contacting a candidate agent with a nucleic acid molecule according to claim 1;
(ii) determining whether said candidate agent modulates the expression of the nucleic acid molecule, such modulation being indicative for a compound useful for the treatment of said ion channel related conditions.

13. A method for identifying an agent modulating the biological activities of a mammalian voltage-gated potassium ion channel, said method comprising the steps
(i) contacting a candidate agent with the mammalian CNA7 polypeptide according to any one of claims 2 to 4; and
(ii) determining whether said candidate agent modulates the biological activities of the said polypeptide.

14. A method for identifying an agent useful for the treatment of diabetes, said method comprising the steps
(i) contacting a candidate agent with a mammalian KCNA7 polypeptide according to any one of claims 2 to 4; and
(ii) determining whether said candidate agent decreases or inhibits the biological activities of the said mammalian KCNA7 polypeptide, such decrease or inhibition being indicative for a compound useful for the treatment of diabetes.

15. A method for identifying an agent useful for the treatment of ion channel related conditions selected from the group consisting of schizophrenia, depression, anxiety attention deficit hyperactivity disorder, migraine, stroke, ischemia, glaucoma, macular degeneration, epilepsy, and neurodegenerative disease, said method comprising the steps
(i) contacting a candidate agent with a mammalian KCNA7 polypeptide according to any one of claims 2 to 4; and
(ii) determining whether said candidate agent modulates the biological activities of the said mammalian KCNA7 polypeptide, such modulation being indicative for a compound useful for the treatment of said ion channel related conditions.

16. A method for the identification of an agent modulating transcription of the human KCNA7 gene, comprising determining whether a candidate agent modulates expression of the human KCNA7 gene via a mechanism dependent upon a regulatory region shown as positions 105 to 114 or 201 to 211 in SEQ ID NO:13.
A method for the identification of an agent modulating transcription of the human KCNA7 gene said method comprising the steps
(i) contacting a candidate agent with a regulatory region shown as positions 105 to 114 or 201 to 211 in SEQ ID NO:13; and
(ii) determining whether said candidate agent modulates expression of the human KCNA7 gene, such modulation being indicative for an agent modulating transcription of the human KCNA7 gene.
